(19) United States
(12) Patent Application Publication
(10) Pub. No.: US 2026/0133167 A1
(43) Pub. Date: May 14, 2026

(54) AUTOMATED CONTINUOUS HDX-MS DEVICE

(71) Applicants: Derek Wilson, King City (CA); Joseph Anacleto, Brampton (CA); Cristina Lento, Toronto (CA)

(72) Inventors: Derek Wilson, King City (CA); Joseph Anacleto, Brampton (CA); Cristina Lento, Toronto (CA)

(21) Appl. No.: 18/859,582

(22) PCT Filed: Oct. 24, 2023
(86) PCT No.: PCT/CA2023/050555

§ 371 Date: Oct. 24, 2024

Related U.S. Application Data
(60) Provisional application No. 63/363,492, filed on Apr. 24, 2022.

(51) Int. Cl. G01N 30/72; G01N 30/24; G01N 33/68

(52) U.S. Cl. CPC G01N 30/7233; G01N 30/24; G01N 33/6848

(57) ABSTRACT

An automated continuous flow system for HDX mass spectrometry, called a continuous flow injection hydrogen deuterium exchange (CFI-HDX), or CFI-TRESI-HDX when a millisecond mixer is incorporated. In some modes, the system can also accommodate automated LC separation, improving sensitivity and allowing a wide range of buffers. Millisecond to hour HDX timepoints can be acquired continuously or discretely in a single experiment, allowing for the detection of conformational/dynamic shifts in target proteins over the full range of structural stabilities and/or binding affinities.

### FIELD OF THE INVENTION

The present specification relates generally to hydrogen deuterium exchange mass spectrometry (HDX-MS), and more particularly to a continuous flow injection time-resolved HDX-MS device which may operate as an automated device.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

Virtually all aspects of protein function and pathogenesis require conformational changes, or shifts in conformational dynamics. Hydrogen deuterium exchange mass spectrometry (HDX-MS) has emerged as a leader among a small number of techniques that allow for characterization of conformational and dynamic shifts with structural resolution, enabling deep explorations of protein activity on the molecular level. In industry, HDX-MS has found ‘high demand’ niches in biotherapeutic characterization and in the determination of protein interaction surfaces, particularly epitopes in antibody/antigen interactions. Interest in this approach has risen to the point where specialized commercial systems are available for automated HDX-MS experiments. These systems are essentially variations on classical LC-MS, combining sophisticated robotic autosamplers for complex mixing and injection programs with refrigerated columns for (acid protease) digestion and reverse phase separation. Commercial platforms have dramatically improved the robustness of HDX-MS measurements, but have several drawbacks including minimum HDX labeling times of around 10 seconds, which limits these systems to well-structured proteins and tight (or at least slow off-rate) protein interactions.

A recurrent theme in recent HDX-MS development has been the need to measure exchange on the ‘complete’ biologically relevant timescale, with labeling times from milliseconds to hours. Millisecond labeling times are of particular interest, since they allow for characterization of dynamic shifts in less-ordered regions of proteins and even ‘intrinsically disordered’ proteins. Several groups have developed devices that combine millisecond rapid mixing with hydrogen deuterium exchange mass spectrometry, including simple capillary setups, microfluidic chips and more recently a stopped-flow setup. However, none of these approaches, with the exception of the microfluidic chip, has yet been used extensively to explore the novel biological phenomena beyond model systems. Part of the reason for this may be that these setups tend to require highly specialized equipment and expertise, and do not incorporate all of the capabilities associated with the commercial systems. For instance, none (with the exception of the nascent stopped-flow system) have demonstrated LC-based, automated workflows.

It would be desirable for an improved HDX-MS system that enables continuous, high throughput analyses with reduced labeling times and experimental time.

Accordingly, there remains a need for improvements in the art.

### SUMMARY OF THE INVENTION

In accordance with an aspect of the invention, there is provided a continuous flow injection time-resolved hydrogen deuterium exchange mass spectroscopy (CFI-TRESI-HDX) system, comprising: a first liquid chromatography (LC) pump operative to provide continuous delivery of protein samples; a second LC pump operative to provide continuous delivery of deuterium (D2O); a capillary mixer comprising an inner capillary and an outer capillary, the inner capillary coupled to the first LC pump and the outer capillary coupled to the second LC pump, the inner capillary and outer capillary in fluid communication via a notch in the inner capillary, the capillary mixer operative to produce a labeled protein sample within a labeling time between 10 milliseconds and 18 seconds, the inner capillary moveable within the outer capillary with the movement of the inner capillary providing adjustment of the labeling time; a quench mixer coupled to the outer capillary, the quench mixer operative to quench the labeled sample with an acid, wherein the output from the quench mixer is suitable for use in hydrogen-deuterium exchange mass spectroscopy.

The movement of the inner capillary may performed at a fixed rate. In one embodiment, the movement of the inner capillary is performed as a continuous fixed rate withdrawal of the inner capillary from the outer capillary. The samples may then be taken continuously with the movement of the inner capillary.

In another embodiment, the movement of the inner capillary is performed via discrete movement of the inner capillary to a plurality of fixed positions relative to the outer capillary. The inner capillary is held each of the fixed positions for a time duration permitting multiple samples to be taken at each fixed position.

The system may further comprise a liquid chromatography (LC) separation system coupled to the quench mixer and operative to receive output from the quench mixer.

In an embodiment, the labeling time is between 150 milliseconds and 10 seconds. In another embodiment, the system performs epitope mapping of the protein sample with a triplicate dataset in less than 90 minutes.

In accordance with another aspect of the invention, there is provided a method of performing continuous flow injection time-resolved hydrogen deuterium exchange mass spectroscopy (CFI-TRESI-HDX), comprising: continuously injecting a protein sample into an inner capillary of a capillary mixer via a first liquid chromatography (LC) pump; continuously injecting a deuterium duffer (D2O) into an outer capillary of the capillary mixer via a second LC pump, the outer capillary and inner capillary in fluid communication via a notch in the inner capillary to produce a labeled sample; moving the inner capillary within the outer capillary to provide adjustment of sample labeling time within the capillary mixer to provide a labeling time between 10 milliseconds and 18 seconds; quenching the labeled sample in a quench mixer coupled to the outer capillary; and outputting the quenched sample for mass spectrographic analysis.

The movement of the inner capillary may performed at a fixed rate. In one embodiment, the movement of the inner capillary is performed as a continuous fixed rate withdrawal of the inner capillary from the outer capillary. The samples may then be taken continuously with the movement of the inner capillary.

In another embodiment, the movement of the inner capillary is performed via discrete movement of the inner capillary to a plurality of fixed positions relative to the outer capillary. The inner capillary is held each of the fixed positions for a time duration permitting multiple samples to be taken at each fixed position.

The method may further comprise passing the quenched sample through a liquid chromatography (LC) separation system coupled to the quench mixer prior to outputting the quenched sample for mass spectrographic analysis.

In an embodiment, the labeling time is between 150 milliseconds and 10 seconds. In another embodiment, the method performs epitope mapping of the protein sample with a triplicate dataset in less than 90 minutes.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a schematic of a TD-HRX reaction chamber;

FIG. 2 is a series of graphs of continuous time point mode of operation on the millisecond to second time scale for the system of FIG. 1;

FIG. 3 is a series of screenshots of outputs from the data analysis module for FIG. 2;

FIG. 4 is a series of graphs of discrete time point mode of operation on the millisecond to second time scale for the system of FIG. 1;

FIG. 5 is a coverage map for Tau using CFI-TRESI-HDX without LC separation;

FIG. 6 is a series of graphs of discrete time point mode of operation with LC separation for the system of FIG. 1; and

FIG. 7 is a series of graphs of discrete and continuous modes of operation for long time point HDX for the system of FIG. 1.

Like reference numerals indicate like or corresponding elements in the drawings.

### DETAILED DESCRIPTION OF THE EMBODIMENTS

The present invention relates generally to an LC-automated system that overcomes many of the barriers to the broader adoption of millisecond HDX-MS and ‘full timescale’ HDX experiments. The system is composed almost entirely LC components, uses autosampler control software, and is capable of multiple modes of automated operation, including discrete and continuous data collection over timescales ranging from milliseconds to hours. Continuous data acquisition may also drastically reduce experiment times, with triplicate measurements up to 1 hour labeling times requiring just under 4 hours of acquisition, although this mode may not allow for the incorporation of reverse-phase chromatography. Data analysis can be carried out using existing commercial or non-commercial HDX software, and for experiments described here, has been automated in a new module incorporated into an existing software suite.

This system may measure HDX kinetics from milliseconds to hours with (effectively) infinite temporal resolution. The result is the ability to explore the full range of protein conformational dynamics with unprecedented precision, from disordered regions and weak binding interactions to structured regions and strong binding interactions.

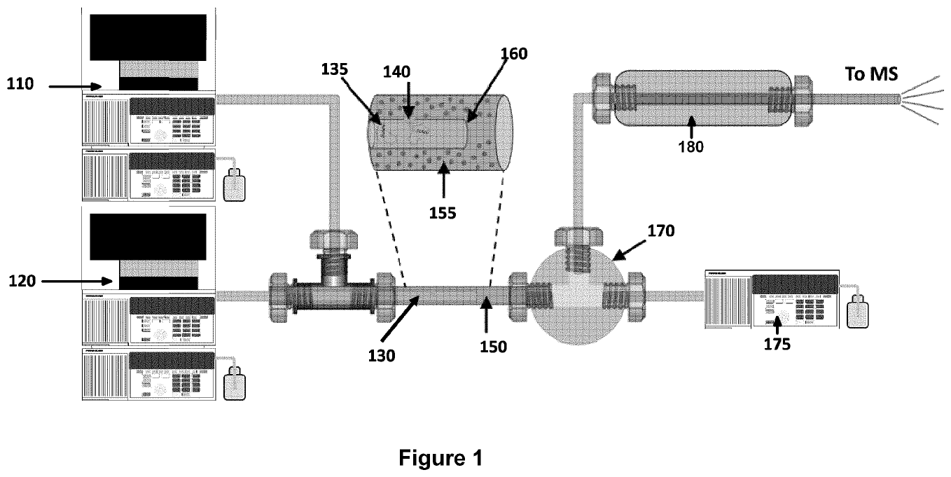

As shown in FIG. 1, the system 100 is based on a capillary mixing system that was first introduced for mass spectrometry applications in 2003. In 2012, that capillary mixing system was incorporated into a microfluidic chip capable of supporting all of the mixing and reaction steps necessary for ‘bottom up’ hydrogen deuterium exchange mass spectrometry. That microfluidic chip setup did not allow for automated operation, making continuous data collection and automated triplicate measurements impossible. The system 100 incorporates a capillary mixer, but the microfluidic chip has been replaced by liquid chromatography (LC) mixing tees and a short PEEK microcolumn (column volumes range from 8 uL to 100 uL, optimized to achieve adequate digestion and reduce carryover) for immobilized acid protease mediated digestion.

The workflow enabled by this device is similar to a classic ‘bottom-up’ HDX approach incorporating acid quenching of the HDX reaction followed by acid protease-based digestion of the labelled protein. Fundamentally, Protein 135 and D₂O 155 are supplied via separate LC pumps (110, 120) with independent autosamplers. The protein mixture 135 passes through a fused silica inner capillary 130 that is blocked at the distal end. Fluid escapes from this capillary through a small notch 140 cut approximately 2 mm from the distal end, forcing the protein solution 135 to mix with D₂O 155 in the narrow inter-capillary space 160 (FIG. 1, inset) between the inner capillary 130 and outer capillary 150. The volume between the end of the inner capillary 130 to the subsequent acid quench mixer 170 may be adjusted by moving the inner capillary 130 within the outer capillary 150, which allows for the acquisition of ‘labelling times’ from 10 milliseconds up to 18 seconds. This adjustment can be made in a continuous manner using a syringe-pump mounted mechanism or in discrete increments by withdrawing the inner capillary 130 from the quench mixer 170, corresponding to the description herein of ‘continuous mode’ and ‘discrete mode’ data acquisition, respectively.

After labeling, the HDX reaction is acid-quenched using a quench mixer 170 (i.e a microLC mixing tee) supplied by an acid pump 175 and the quenched solution is passed through an acid-protease column 180 to generate the labeled peptides whose deuterium uptake will be analyzed. In continuous mode, the peptides are injected directly into the mass spectrometer, resulting in a minimum ‘quench-to-ESI’ delay of 8 to 60 s, depending on the digest column volume. This short period is generally insufficient for significant back-exchange to occur, with typical back exchange levels staying below 5%. In discrete mode, peptides can either be directly injected into the mass spectrometer or passed into a reversed phase separation module. The latter approach significantly enhances coverage and redundancy, but also drastically increases the experiment time and back exchange, bringing both to levels consistent with known systems.

A series of experiments were conducted to determine the optimal solutions and flow conditions for this device, with the aim of minimizing back exchange and carryover, while maximizing quench conditions and sequence coverage. The experimental details of this optimization are provided in Table 1. In discrete mode, conventional quenching solutions, like 5%-10% acetic acid in water, were found to allow significant retention and carryover of peptides from previous replicates on the agarose beads of the acid protease column. In continuous mode, this retention was observed as a linear increase in deuterium uptake superimposed on the ‘true’ uptake kinetics. Ultimately, it was determined that carryover and drift could be essentially eliminated by adding ammonium formate to the quench buffer (while maintaining an appropriate quench pH with formic acid), resulting in the following optimized buffer conditions for cytochrome C and Tau proteins: 100 mM ammonium acetate protein buffer (pH 7.0), and 3% formic/40 mM ammonium formate as the quench buffer (pH 2.3).

TABLE 1
CFI-TRESI Buffers and Carryover

| | | Final | Digest |
|---|---|---|---|
| Quench Buffer | Protein Buffer | pH | Carryover |
| 3% Formic, 40 mM Amm. Formate (pH 2.4) | 50 mM Amm. Acetate (pH 7.0) | 2.5 | Negligible |
| 3% Formic, 40 mM Amm. Formate (pH 2.4) | 100 mM Amm. Acetate (pH 7.0) | 2.5 | Negligible |
| 1% Formic Acid (pH 2.2) | 100 mM Amm. Acetate (pH 7.0) | 2.5 | High |
| 2% Formic, 25 mM Amm. Formate (pH 2.3) | 50 mM Amm. Acetate (pH 7.0) | 2.5 | Moderate |
| 2% Formic, 25 mM Amm. Formate (pH 2.3) | 100 mM Amm. Acetate (pH 7.0) | 2.6 | Moderate |
| 10% Acetic Acid (pH 2.2) | 100 mM Amm. Acetate (pH 7.0) | 2.6 | High |
| 1% Formic Acid (pH 2.2) | 250 mM Amm. Acetate (pH 7.0) | 2.8 | Moderate |
| 19% Formic, 25 mM Amm. Formate (pH 2.6) | 100 mM Amm. Acetate (pH 7.0) | 2.8 | Moderate |
| 1% Formic, 25 mM Amm. Acetate (pH 2.7) | 100 mM Amm. Acetate (pH 7.0) | 2.9 | Moderate |
| 10% Acetic Acid (pH 2.2) | 250 mM Amm. Acetate (pH 7.0) | 3.0 | Moderate |
| 10% Acetic, 25 mM Amm. Acetate (pH 2.8) | 100 mM Amm. Acetate (pH 7.0) | 3.1 | Moderate |

Millisecond HDX, Continuous mode. In most recent conventional HDX studies, uptake kinetics are treated as a ‘secondary’ result, with the ‘headline’ number being the total uptake difference between protein states (e.g., ‘bound’ and ‘unbound’) over all timepoints measured. This is understandable given the unequivocal nature of the ‘summed difference’ parameter in defining binding sites or regions that undergo substantial dynamic shifts. However, the HDX kinetics themselves can provide critical information, including, for example, whether an observed difference in deuterium uptake between two protein states is due to a change in ‘structure’ or a change in ‘dynamics’ or both. There is also evidence that careful analysis HDX kinetics can reveal kinetic and thermodynamic parameters (i.e., K_{d} and k_{off}) for binding interactions.

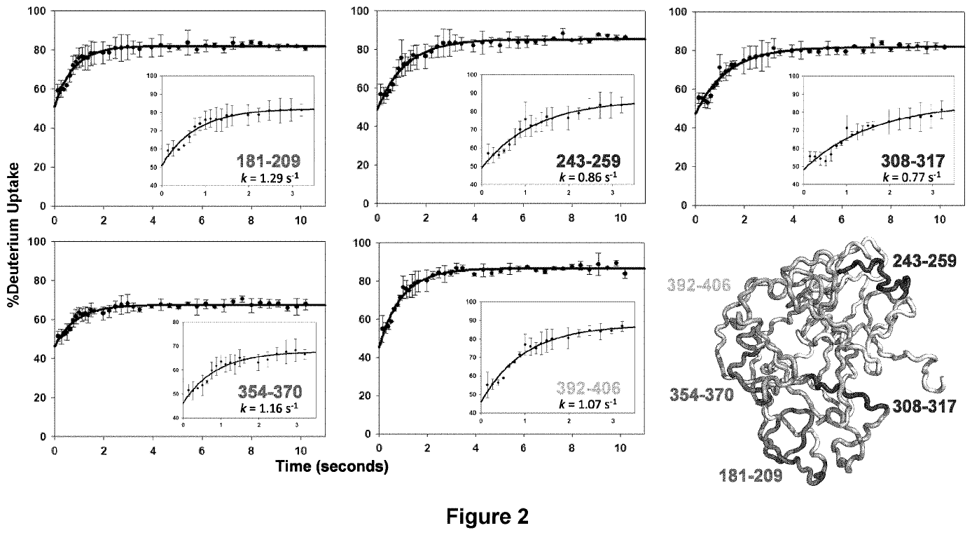

One reason that careful analysis of HDX kinetics may not be widely practiced may be that the acquisition of sufficient time-points to accurately determine the observed uptake rate constant would be a lengthy and arduous process. In millisecond continuous mode acquisition in the present system 100, the inner capillary 130 is continuously withdrawn from the end of the outer capillary 150 at a given rate while HDX data are continuously collected. This continuous withdrawal and collection allows for an essentially unlimited number of HDX timepoints to be acquired, typically from 150 milliseconds to 10 seconds, with the rate of withdrawal, scan time and the width of the TIC segments selected by the user ultimately determining the number and spacing of ‘timepoints’ used in the analysis. Time points as low as 10 milliseconds or of 15 seconds or longer may be achieved using the system. An example of this type of analysis is provided in FIG. 2, using Tau protein as a model. Five representative Tau peptides were analyzed from a continuous pullback experiment spanning 140 ms to 10 s. Peptide sequences and rate constants are listed in the insets, with a zoomed in view of the first 3.5 seconds where most of the exchange is taking place. Error bars represent one standard deviation from two replicates. By fitting the data to a single exponential expression, it may be possible to precisely extract ‘observed’ (phenomenological) rate constants, identifying regions that are weakly protected from exchange due to loci of residual structure in the Tau conformational ensemble.

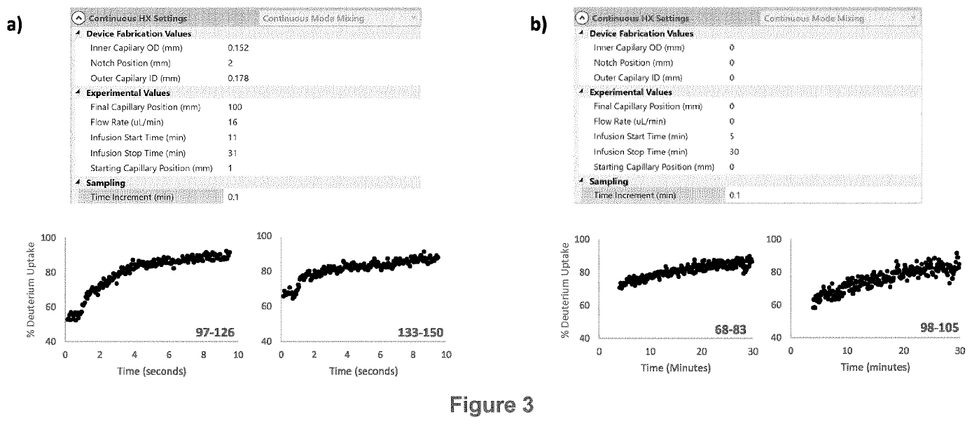

Another potential advantage of millisecond continuous mode data acquisition is the exceedingly short experiment time, owing to the fact that individual timepoints are not independent runs in this mode. In principle, a complete triplicate dataset with unlimited timepoints over the full 150 ms to 10 s timecourse could be acquired in just over 90 minutes, with user intervention required only between replicates to reset the position of the capillary mixer. Manual analysis of these data can be taxing, requiring the user to select n equally broad segments of the ‘chromatogram’—corresponding to the desired number of timepoints—and then individually analyze each of them for every peptide's uptake. To assist with this process, a new software module was generated for the Mass Spec Studio 2.0 software package that automates data extraction and generation of kinetic plots for each identified peptide directly from continuous-mode data. Screenshots and outputs from this module are provided in FIG. 3. FIG. 3(a) shows an analysis of continuous millisecond to second data for representative Tau peptides. In this mode the reaction occurs in the capillary-based mixing device with the listed parameters. The delay in uptake observed during the first 140 ms of the curve is a result of the deadtime between start of the pullback to the ESI source. FIG. 3(b) shows an analysis of continuous long time point data for representative Cyt c peptides. In this mode, the reaction occurs in the autosampler and does not depend on the parameters of the capillary-based mixing device.

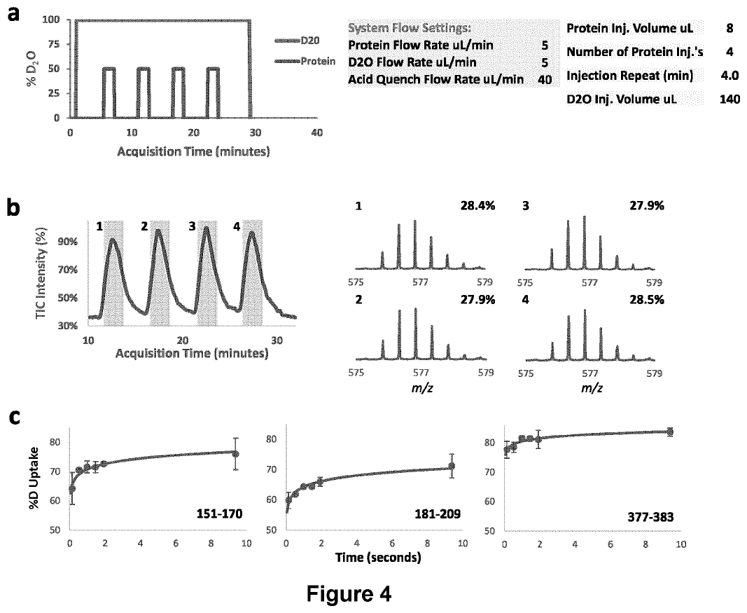

Millisecond HDX, Discrete Mode. Millisecond discrete mode is used to acquire a limited number of HDX time points typically between 150 ms and 10 s, with automated technical replicates. The main advantage of this mode is that it allows for indefinite-length acquisitions at individual HDX timepoints (subject to limitations on sample consumption), and can incorporate LC-separation. In terms of workflow, the defining characteristic of this mode is that the inner capillary is held at a fixed position for multiple injections, each corresponding to a technical replicate of a given timepoint. When the desired number of technical replicates have been acquired, the inner capillary is manually withdrawn a fixed distance within the outer capillary to transition to a new HDX timepoint. To facilitate autosampler programming in this mode, we created an excel worksheet tool CFIset that estimates sample elution times and visualizes the experiment. A typical millisecond discrete mode experiment is shown in FIG. 4, with predicted and actual elutions using cytochrome c as a model.

The CFIset profile (FIG. 4a) is idealized in the sense that it assumes perfect ‘plug-flow’ and no peak broadening due to mixing in the labelling and quenching steps, and thus narrow square-pulse shaped injections. Nonetheless, the tool successfully predicts the signal onset time and approximate peak width for various flow conditions. Actual injection profiles, like the one shown in FIG. 4b, exhibit the expected normal-skewed square distributions reflecting laminar flow and mixing within the device. FIG. 4b also shows a sample peptide (a.a. 96-105) extracted from these replicates for the 240 ms exchange timepoint, demonstrating inter-injection reproducibility.

Exchange kinetics may be acquired in this mode by manually withdrawing the inner capillary within the outer capillary after the desired number of replicate injections at the current timepoint have been performed. For labeling times on the milliseconds to second timescale, complete exchange kinetics profiles for all peptides (e.g., FIG. 4c) can typically be acquired in triplicate within 3 hours for six timepoints (i.e., 30 min per timepoint). The experimental rapidity of this technique is understood to be a result of (i) the HDX short labeling times, (ii) automated replicate acquisition and (iii) the lack of LC separation. It has been previously noted through experimental work that short (millisecond) HDX labeling times are sensitive to the types of interactions covered by conventional time-scale HDX and also offer substantial advantages in characterizing disordered regions, weak binding interactions and allosteric effects. However, the inability to include LC separation in these experiments has been a substantial limitation, particularly since it has prevented the use of the non-volatile buffers in which some proteins are maximally soluble. The ‘injection pulse’ nature of millisecond discrete mode CFI-TRESI-HDX allows for automated online LC separation, which is carried out while the HDX mod-ule of the apparatus is being washed. The incorporation of LC into CFI-HDX experiments is discussed in detail below.

Incorporation of Liquid Chromatography Separations. Liquid chromatography (LC) has long been used in ‘bottom-up’ HDX workflows, and is incorporated into known HDX systems. The use of LC has several substantial advantages over HDX experiments that do not incorporate LC separation, notably increased sensitivity and sequence coverage, and a much-improved tolerance for ‘biologically relevant’ non-volatile buffer salts. The latter advantage can be critically important when dealing with proteins that aggregate in ‘ESI-friendly’ solvent alternatives like NH4Ac ‘buffers’. However, LC separation also comes with notable disadvantages resulting from the length of time required for effective separation/elution on a reverse-phase column (typically 5-20 minutes). In particular, this delay between HDX quenching and ionization substantially increases back exchange, whose minimization has recently been a major focus of conventional HDX method development.

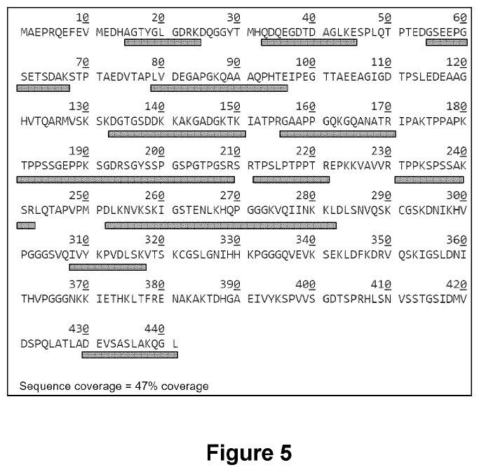

The use of LC separation also precludes continuous mode data acquisition workflows and, even for the shortest gradients, greatly increases the experiment time. For example, a triplicate, 5 time-point HDX run with a maximum 2 hour labelling time could easily require 24 hours or more of continuous measurement. In long-timepoint discrete mode, the CFI-HDX system reduces this considerably; a similar experiment to the one described above would require roughly 12 hours. In millisecond discrete mode, the total experiment time corresponds to the width of the HDX timepoint pulse/wash sequence, times the number of replicates, times the number of timepoints, or a reduction down to roughly 4 hours for the experiment described above. In spite of these drawbacks, the advantages of LC separation in terms of sequence coverage/redundancy and broad applicability of the method are substantial (FIG. 5). For these experiments, a 50:50 pepsin:protease XIII column was used with quenching conditions of using 3% FA+40 mM ammonium acetate (pH=2.4). A sequence coverage of 47% was observed.

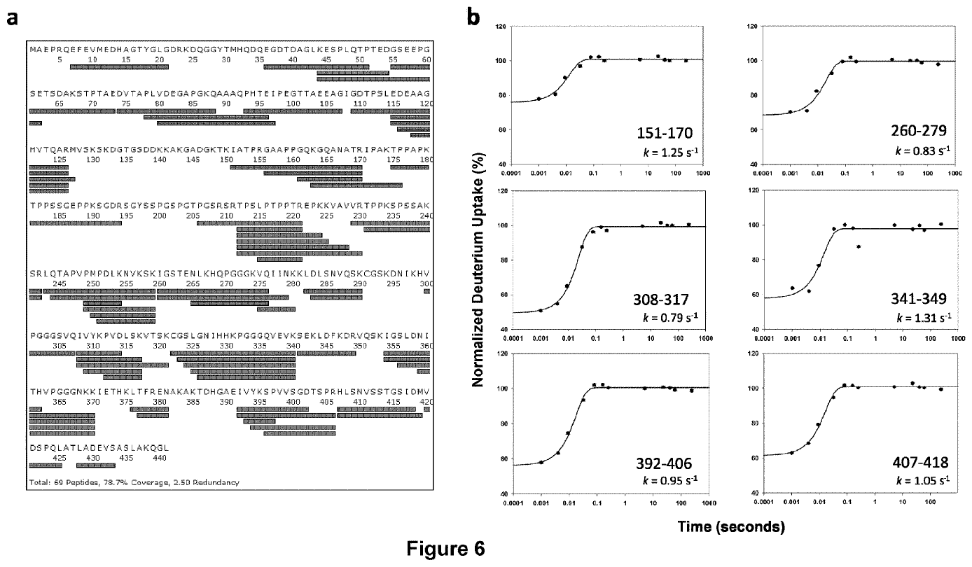

A typical CFI-TRESI HDX analysis with LC, incorporating timepoints from milliseconds to hours and using Tau as a model, is shown in FIG. 6. While this experiment provides an accurate measurement of uptake rates across the entire HDX window (including tens of hours if so desired), the increase in back exchange was substantial and ranged considerably for each peptide (from ˜30%-40%) in a way that did not correlate with retention time. On the other hand, sequence coverage improved from 47% to 79% and redundancy from 0-2.5 (comparing FIG. 6a to FIG. 5, it is noted that Tau is a challenging target for pepsin and pXIII due to high positive charge). Importantly, the measured uptake rates were in excellent agreement with those measured in millisecond continuous mode (comparing FIG. 6b to FIG. 2).

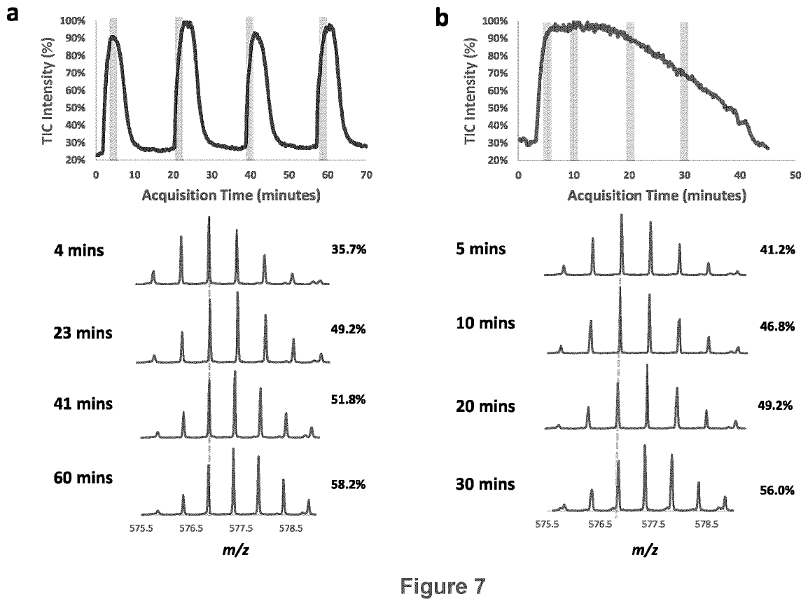

Conventional timescale discrete and continuous modes. The ‘conventional’ timescale for HDX experiments, corresponding to 10 seconds to several hours of labeling time, is useful for characterizing tight binding interactions and conformational dynamics in ‘structured’ regions of proteins. The system 100 is further capable of automated conventional timescale HDX measurements, with labelling times from 4 minutes to tens of hours, which is achieved by mixing in the autosampler and bypassing the capillary mixer for injection. Injection of the mixed solution in this manner can be continuous, allowing for unlimited timepoint acquisition, or pulsed (discrete mode), allowing for LC separation. Equivalent continuous mode experiments have occasionally been attempted in the past, while the discrete mode with LC separation is essentially equivalent to the conventional known systems currently in wide-spread use. Typical data from discrete and long timescale measurements are shown in FIG. 7.

For the discrete mode experiment (FIG. 7a), deuterium uptake on either end of the peak profiles is impacted by dilution of the labelling solution with carrier solution. To avoid this, data are analyzed only from where the peak hits 80% of its maximum height, where dilution effects are negligible. FIG. 7a shows TIC and mass spectra of a representative Cyt c peptide (a.a. 96-105) during discrete long time point acquisition and FIG. 7b shows TIC and mass spectra of a representative Cyt c peptide (a.a. 96-105) during continuous long time point acquisition. In both modes, the exchange reaction is initiated at the start of the acquisition file.

Note that since conventional timescale measurements can be carried out without any modification of the apparatus, these experiments can be initiated (with limited user intervention) immediately following a millisecond run, allowing for acquisition of the ‘full’ HDX timecourse in a single experiment. The same benefits and limitations apply as for millisecond studies: Continuous measurements require the use of ‘electrospray friendly’ buffers and have lower sensitivity but generate an unlimited number of timepoints in a comparatively very short experiment time (corresponding to slightly longer than 3× the longest label-ling time for triplicate data collection). Discrete measurements require substantially more experiment time, but are compatible with LC, which has a number of advantages and limitations (see above).

### EXPERIMENTAL SECTION

Materials. Cytochrome c (C7752) from equine heart (299%), deuterium oxide (D2O, 99.99%), ammonium acetate (≥98%) and formic acid (≥98%), were purchased from Sigma-Aldrich (St. Louis, MO). Tau protein was expressed from E. coli BL21 cells containing a pET-29b vector encoding the htau40 isoform. Purification was carried out as and protein stored in 20% glycerol at −80° C. Both cytochrome c and tau were resuspended or buffer exchanged into 50 mM ammonium acetate prior to MS analysis.

Full time-scale HDX experiments. CFI-TRESI-HDX experiments were completed according to the experimental setup shown in FIG. 1. PerkinElmer Series 200 pumps and autosamplers were used to deliver solvents. Pepsin (porcine gastric mucosa, Sigma-Aldrich) or Protease XIII (Aspergillus saitoi, Sigma-Aldrich) was cross-linked in-house onto NHS-activated agarose (Pierce™, Thermo Fisher Scientific). Digestion columns were constructed in-house using PEEK tubing with an ID of 0.040″ and a 2 μm pore-size frit upstream of the ESI emitter. All data was acquired on a Waters Synapt G2-S, IMS was employed in the TriWave cell to improve spatial resolution of peptides in the digested samples. Peptide identification was performed using Proteome Discoverer (Thermo Fisher Scientific) after LC-MS/MS analysis with the Orbitrap Elite Hybrid Ion Trap-Orbitrap Mass Spectrometer. Identified peptides were analyzed for deuterium uptake using Mass Spec Studio 2.0.

Full time-scale HDX experiments with LC separation. For CFI-TRESI-HDX coupled to LC separation, samples were directly loaded into a cooled HDX-UPLC system (Waters, Mil-ford, MA) with digestion on an in-house built pepsin/proteaseXII column at 15° C. and desalting at 0° C. The flowrate during digestion was 8 μL/min protein, 8 μL/min D2O, and 64 μL/min acid quench. Desalting took place at 80 μL/min for one minute and ramped up to 140 uL/min for 2 minutes. Peptides were separated by reverse phase chromatography (Waters Acquity BEH C18, 1.7 μm, 1 mm×100 mm) with a 7-minute gradient from 5% to 35% using acetonitrile with 0.1% formic acid at 35 μL/min.

Data Analysis. Continuous uptake measurement data were analyzed using a new ‘continuous measurement’ HX module developed for for Mass Spec Studio 2.0. This module was built as an extension to the classic peptide HX-MS module (HX-DEAL) in order to reuse the existing model-based deuterium uptake calculations and apply them on data acquired using the continuous pullback method. A custom processing routine was created (‘Continuous HX-MS’) which aggregates MS1 data over the pre-defined ion-mobility (IM) range of each peptide and measures deuterium uptake at continuous intervals in time. During data processing, the measured retention times in the data file are transformed to the true HX reaction times by applying the experimental and hardware specifications in the parameters section of the new processing routine (e.g. time increment, flow rate, notch position, start/stop infusion time, etc.). The continuous % D values for each peptide as well as their transformed reaction times are exportable in .csv format via the ‘raw’ output option inside the Export Wizard.

It should also be noted that the steps described in the method of use can be carried out in many different orders according to user preference. The use of “step of” should not be interpreted as “step for”, in the claims herein and is not intended to invoke the provisions of any statutory requirements. It should also be noted that, under appropriate circumstances, considering such issues as design preference, user preferences, marketing preferences, cost, structural requirements, available materials, technological advances, etc., other methods are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the Patent and Trademark Office and the public generally, and especially the scientist, engineers and practitioners in the art who are not familiar with patent or legal terms or phraseology, to determine quickly from a cursory inspection the nature and essence of the technical disclosure of the application.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. A continuous flow injection time-resolved hydrogen deuterium exchange mass spectroscopy (CFI-TRESI-HDX) system, comprising:
a first liquid chromatography (LC) pump operative to provide continuous delivery of protein samples;
a second LC pump operative to provide continuous delivery of deuterium (D₂O);
a capillary mixer comprising an inner capillary and an outer capillary, the inner capillary coupled to the first LC pump and the outer capillary coupled to the second LC pump, the inner capillary and outer capillary in fluid communication via a notch in the inner capillary, the capillary mixer operative to produce a labeled protein sample within a labeling time between 10 milliseconds and 18 seconds, the inner capillary moveable within the outer capillary with the movement of the inner capillary providing adjustment of the labeling time;
a quench mixer coupled to the outer capillary, the quench mixer operative to quench the labeled sample with an acid,
wherein the output from the quench mixer is suitable for use in hydrogen-deuterium exchange mass spectroscopy.

2. The system of claim 1, wherein the movement of the inner capillary is performed at a fixed rate.

3. The system of claim 2, wherein the movement of the inner capillary is performed as a continuous fixed rate withdrawal of the inner capillary from the outer capillary.

4. The system of claim 3, wherein the samples are taken continuously with the movement of the inner capillary.

5. The system of claim 2, wherein the movement of the inner capillary is performed via discrete movement of the inner capillary to a plurality of fixed positions relative to the outer capillary.

6. The system of claim 5, wherein the inner capillary is held each of the fixed positions for a time duration permitting multiple samples to be taken at each fixed position.

7. The system of claim 1, further comprising a liquid chromatography (LC) separation system coupled to the quench mixer and operative to receive output from the quench mixer.

8. The system of claim 1, wherein the labeling time is between 150 milliseconds and 10 seconds.

9. The system of claim 4, wherein the system performs epitope mapping of the protein sample with a triplicate dataset in less than 90 minutes.

10. A method of performing continuous flow injection time-resolved hydrogen deuterium exchange mass spectroscopy (CFI-TRESI-HDX), comprising:
continuously injecting a protein sample into an inner capillary of a capillary mixer via a first liquid chromatography (LC) pump;
continuously injecting a deuterium duffer (D2O) into an outer capillary of the capillary mixer via a second LC pump, the outer capillary and inner capillary in fluid communication via a notch in the inner capillary to produce a labeled sample;
moving the inner capillary within the outer capillary to provide adjustment of sample labeling time within the capillary mixer to provide a labeling time between 10 milliseconds and 18 seconds;
quenching the labeled sample in a quench mixer coupled to the outer capillary; and
outputting the quenched sample for mass spectrographic analysis.

11. The method of claim 10, wherein the movement of the inner capillary is performed at a fixed rate.

12. The method of claim 11, wherein the movement of the inner capillary is performed as a continuous fixed rate withdrawal of the inner capillary from the outer capillary.

13. The method of claim 12, wherein the outputting of the quenched samples is performed continuously with the movement of the inner capillary.

14. The method of claim 10, wherein the movement of the inner capillary is performed via discrete movement of the inner capillary to a plurality of fixed positions relative to the outer capillary.

15. The method of claim 14, wherein the outputting of the quenched samples is performed for multiple samples at each fixed position.

16. The method of claim 10, further comprising passing the quenched sample through liquid chromatography (LC) separation prior to outputting the quenched sample for mass spectrographic analysis.

17. The method of claim 10, wherein the labeling time is between 150 milliseconds and 10 seconds.

18. The method of claim 13, wherein the method performs epitope mapping of the protein sample with a triplicate dataset in less than 90 minutes.
